(19) United States
(12) Patent Application Publication
(10) Pub. No.: US 2025/0114583 A1
(43) Pub. Date: Apr. 10, 2025

(54) Enhanced Sclerotomy Port with Sensory System for Eye Position and Orientation Tracking in Intraocular Surgery Enabling Augmented Reality Applications and Automated Surgical Systems

(71) Applicant: Run Zhou Ye, Rochester, MN (US)

(72) Inventors: Run Zhou Ye, Rochester, MN (US); En Zhou Ye, Montreal (CA); En Hui Esther Ye, Montreal (CA); Joseph Ye, Montreal (CA); Jacob Ye, Montreal (CA); Bin Zhang, Montreal (CA); Ming Ye, Montreal (CA)

(21) Appl. No.: 18/814,516

(22) Filed: Aug. 25, 2024

(51) Int. Cl. A61M 39/02; A61B 17/34; A61F 9/007

(52) U.S. Cl. CPC A61M 39/02; A61F 9/007; A61B 17/3423; A61M 2205/3313; A61M 2205/502

(57) ABSTRACT

A novel sclerotomy port equipped with an integrated sensory system that enhances intraocular surgical precision by providing real-time eye position and orientation data. This port supports augmented reality applications and automated surgical systems, offering dynamic visual aids for improved procedural outcomes.

### FIELD OF THE INVENTION

This invention relates to the field of medical devices for ophthalmic surgery, specifically to an advanced sclerotomy port designed to facilitate precise intraocular operations through real-time tracking of eye position and orientation, enabling integration with augmented reality technologies and automated surgical systems.

### BACKGROUND OF THE INVENTION

Problem Statement: Intraocular surgeries require exceptionally precise manipulations within the eye's limited space. Traditional sclerotomy ports provide physical access for surgical instruments but lack any means to track the dynamic position or orientation of the eye.

Prior Art Limitations: Existing sclerotomy ports do not offer real-time integration with visualization technologies, which may lead to better surgical outcomes if implemented.

Need for the Invention: There is a need for a sclerotomy port that not only allows surgical instrument access but also enhances the ability of surgeons and automated systems to perform precise maneuvers by providing real-time data on eye movement and orientation, interfacing seamlessly with augmented reality systems or automated surgical systems to improve visual guidance during surgery.

Summary of the Invention: This invention addresses these needs by providing a sclerotomy port equipped with an integrated or externally attached sensory system comprising an accelerometer, gyroscope, and additional orientation sensors. This system is designed to monitor the position and orientation of the eye continuously and convey this information to an augmented reality interface or automated surgical system, thereby enhancing the ability to perform precise and safe intraocular procedures.

### BRIEF DESCRIPTION OF THE DRAWINGS

FIG. 1: This figure illustrates an embodiment of the sclerotomy port with all sensors integrated within the body of the port. The cylindrical body (101) houses the accelerometer (102), gyroscope (103), magnetometer (104), and additional components for sensor data transmission or a battery (105). The integration of these sensors within the port body ensures seamless tracking of eye position and orientation during surgery.

FIG. 2: This figure shows an alternative embodiment where the sensors (accelerometer [203], gyroscope [204], and magnetometer [205]) and additional components for sensor data transmission or a battery (105) are enclosed within an external casing (202) attached to the body (201) of the sclerotomy port. This design allows for flexible sensor placement and easier maintenance or replacement of the sensory components while maintaining biocompatibility and surgical functionality.

FIG. 3: This figure depicts an embodiment of the sclerotomy port with wired data transmission capabilities. Wires (306) extend from the port body, connecting the integrated sensors (accelerometer, gyroscope, magnetometer) to an external data processing unit. This setup is designed for environments where wireless transmission is not feasible or where real-time wired connections are preferred for data reliability.

FIG. 4: Similar to FIG. 2, this figure shows an embodiment with an externally attached casing housing the sensors, but with wires (407) extending out of the casing for wired data transmission and/or power supply to the device. This configuration provides an alternative method for ensuring continuous operation during surgery, particularly in settings where external power is needed.

FIG. 5: This figure illustrates the operational flow of the sclerotomy port system. It begins with the surgical insertion of the port into the scleral wall (501), followed by the activation of the integrated sensors (502). These sensors capture real-time data on eye position and orientation (503), which is then transmitted (504) to an external processing unit (505). The flowchart also depicts the integration of data from multiple ports to enhance monitoring accuracy during surgery (506).

### DETAILED DESCRIPTION OF THE INVENTION

### Detailed Construction

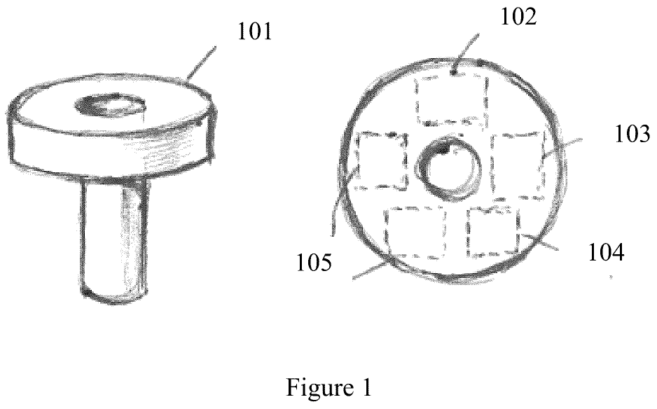

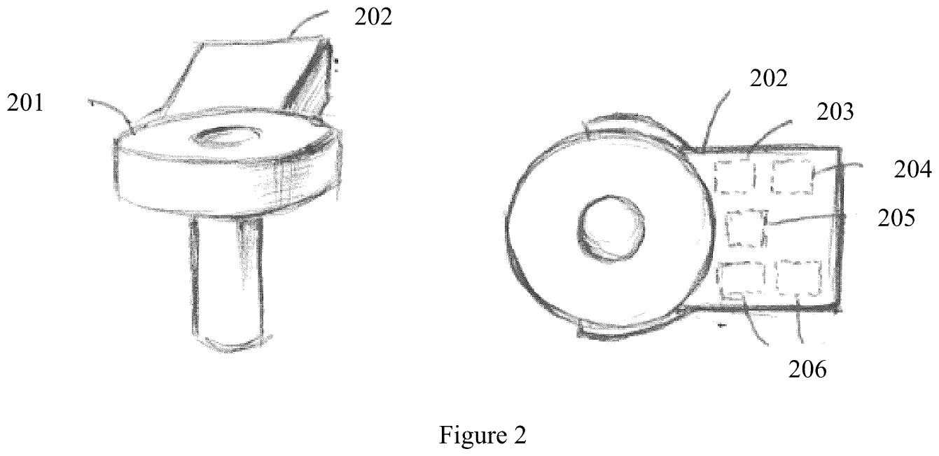

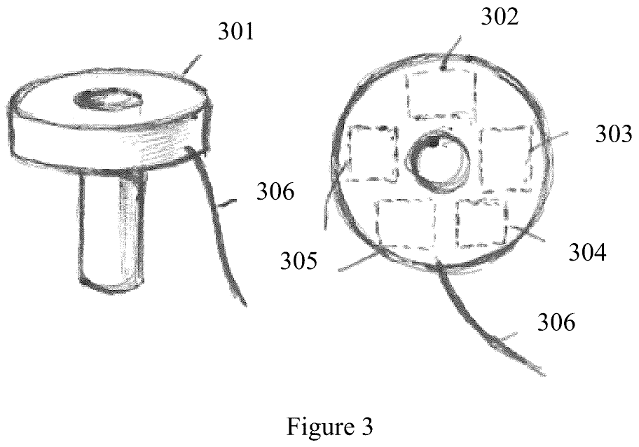

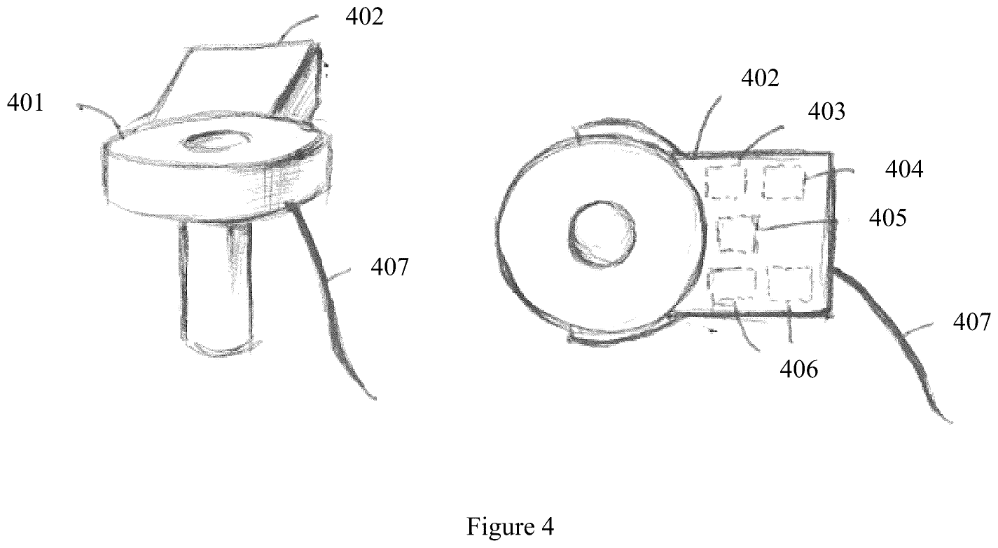

Sclerotomy Port: Constructed from biocompatible materials, the port features a cylindrical body with a central lumen to accommodate surgical instruments. The external surface is ergonomically designed to facilitate easy insertion and stable placement within the scleral tissue. Referring now to FIG. 1, the sclerotomy port comprises a cylindrical body (101), sensors including an accelerometer (102), a gyroscope (103), a magnetometer (104), and other chips for sensor data transmission or battery (105). FIG. 1 shows one embodiment with all sensors integrated within the body of the sclerotomy port. FIG. 2 shows on embodiment where the sensors lie within a casing that is attached to the body of the sclerotomy port. FIGS. 3 and 4 show embodiments with wires extending out of the device for wired data transmission and/or power supply to the device.

Sensor Integration: Positioned within the walls of the port (FIGS. 1 and 3) or attached externally (FIGS. 2 and 4), the sensors (accelerometer, gyroscope, and orientation sensors) are encapsulated in a biocompatible housing to prevent interference with the surgical environment while capturing accurate data.

Data Processing Unit: A unit that receives, processes, and translates sensor data into usable information for AR systems or automated surgical controls.

Communication Module: Capable of transmitting the collected data either wirelessly or via wired connections to ensure flexibility in various surgical environments.

### Operational Methodology

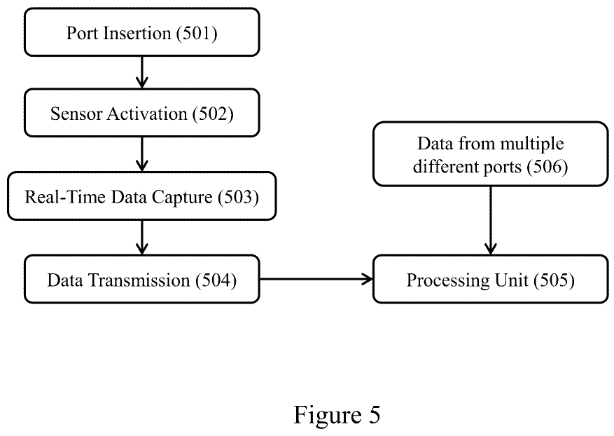

Port Insertion and Activation: As illustrated in FIG. 5, the sclerotomy port is surgically inserted into the scleral wall (501). Once inserted, the port is activated, and the sensors initiate real-time monitoring (502).

Continuous Data Capture and Transmission: The sensors continuously capture real-time data on the eye's position and orientation (503). This data is then transmitted to an external processing unit (504) via either wired or wireless communication, depending on the port's configuration.

Multi-Port Setup: If multiple sclerotomy ports are used, each port transmits its data to the processing unit, where the data is integrated to enhance the accuracy of eye tracking during surgery (505).

Augmented Reality and Automated Surgery Integration: Processed data is relayed to an augmented reality system or automated surgical system that overlays dynamic, real-time surgical guides and eye position indicators over the field of view, enhancing spatial awareness and procedural accuracy.

1. A surgical port device for insertion into the scleral wall during intraocular surgery, the device comprising: a body; at least one sensor configured to detect at least one of position or orientation with a predefined accuracy, wherein the at least one sensor is operatively associated with the body, either integrated within the body or attached thereto; a communication module integrated within the body or attached thereto, capable of transmitting data generated by the at least one sensor to an external receiver via wireless or wired transmission methods.

2. The surgical port device of claim 1, wherein the at least one sensor includes an accelerometer.

3. The surgical port device of claim 1, wherein the at least one sensor includes a gyroscope.

4. The surgical port device of claim 1, wherein the at least one sensor includes a magnetometer.

5. The surgical port device of claim 1, wherein the at least one sensor further includes an infrared sensor.

6. The surgical port device of claim 1, wherein the communication module is capable of both wireless and wired data transmission.

7. The surgical port device of claim 1, further comprising a data processing unit operatively connected to the at least one sensor for processing data, capable of interfacing with both wireless and wired communication modules.

8. The surgical port device of claim 7, wherein the data processing unit is configured to generate real-time feedback based on the processed data.

9. The surgical port device of claim 8, wherein the real-time feedback includes visual, auditory, or haptic feedback.

10. The surgical port device of claim 1, further comprising an augmented reality interface that receives data from the communication module and displays augmented reality visualizations based on the data, adaptable to receive data via both wireless and wired transmissions.

11. The surgical port device of claim 10, wherein the augmented reality visualizations provide a representation of surgical instruments relative to intraocular structures.

12. The surgical port device of claim 1, wherein the body is cylindrical and sized to facilitate insertion into the scleral wall without significant tissue damage.

13. The surgical port device of claim 1, wherein the device is configured to operate continuously throughout the duration of the surgical procedure.

14. The surgical port device of claim 1, wherein the at least one sensor is encapsulated within a biocompatible material that is compliant with medical standards for intraocular use.
